SEQUENCE LISTING (7 sequences)

SEQ ID NO: 1 - protein, 16 aa, Homo sapiens
AAGDVDIGDAAYYFER

SEQ ID NO: 2 - protein, 16 aa, Homo sapiens
ALGSGMAVDCSTLTSK

SEQ ID NO: 3 - protein, 17 aa, Homo sapiens
FVAAVHYEQPTIQIELR

SEQ ID NO: 4 - protein, 12 aa, Homo sapiens
MTVCSPDGPGGR

SEQ ID NO: 5 - protein, 8 aa, Homo sapiens
HHILIDLR

SEQ ID NO: 6 - protein, 8 aa, Homo sapiens
GESLFQGR

SEQ ID NO: 7 - protein, 8 aa, Homo sapiens
GEPLQVER

(19) United States
(12) Patent Application Publication
(10) Pub. No.: US 2015/0037824 A1
(43) Pub. Date: Feb. 5, 2015

(54) URINARY BIOMARKER FOR URINARY TRACT CANCER AND APPLICATION OF THE SAME

(71) Applicant: CHANG GUNG UNIVERSITY, TAO-YUAN CITY (TW)

(72) Inventors: YI-TING CHEN, TAO-YUAN (TW); JAU-SONG YU, TAO-YUAN (TW); CHIEN-LUN CHEN, TAO-YUAN (TW); YU-SUN CHANG, TAO-YUAN (TW)

(73) Assignee: CHANG GUNG UNIVERSITY, TAO-YUAN CITY (TW)

(21) Appl. No.: 14/057,285

(22) Filed: Oct. 18, 2013

(30) Foreign Application Priority Data
Apr. 12, 2013 (TW) 102113166

(51) Int. Cl. G01N 33/574

(52) U.S. Cl. CPC G01N 33/57438; G01N 33/57407; G01N 2333/705
USPC 435/7.94; 436/501; 435/7.92; 436/64; 530/350

(57) ABSTRACT

A urinary biomarker for urinary tract cancers and applications of the same are revealed. TACSTD2 is used as a non-invasive urinary biomarker for urinary tract cancers due to a feature that the TACSTD2 protein is increased significantly in urine of patients with urinary tract cancers. The quantitative urinary biomarker shows high specificity and high sensitivity for urinary tract cancer detection. Besides increasing screening efficiency, early diagnosis and early treatment of urinary tract cancers, the biomarker can also be used to assess malignancy of urinary tract cancers and monitor tumor progression for determining optimal treatment against the disease and improving the treatment results.

### BACKGROUND OF THE INVENTION

1. Fields of the invention

The present invention relates to a urinary biomarker, especially to a urinary biomarker for assessing urinary tract cancers and applications of the same.

2. Descriptions of Related Art

According to statistics of Cancer Statistics 2013 urinary tract cancers are among the most common cancers in the United States. According to the most recent estimates of the American Cancer Society, there will be 72,570 new cases of bladder cancer in the United States and 15,210 deaths from bladder cancer in 2013. Prostate cancer, bladder cancer and kidney cancer are most common in men while leading cancers in women are bladder cancer and kidney cancer. Among urinary tract cancers, the mortality of bladder cancer is the second high and is increasing year by year. Patients with kidney cancer being detected at early stage are of clinical important. For patients at early stage, surgical resection offers the only chance for cure. After surgery, the five-year survival rate is as high as 80%. Chemotherapy, immunotherapy and target therapy are recommended for late-stage kidney cancer patients. Yet the prognosis is poor and the five-year survival rate is only 22%. Thus early detection is critical in the fight against urinary tract cancers. Although many bladder cancer biomarkers have been detected and reported, none of them have shown sufficient sensitivity and specificity. In recent years, there is a trend to discover biomarkers for diseases in body fluids. As a non-invasive specimen, urine is used to find out tumor-derived molecules released directly from urinary tract system. The specific tumor-derived molecules have great potential in evaluating initiation and progression of urinary tract cancers clinically.

The main test methods for bladder canner used in clinic are as follows:

(1) Bloody urine: bloody urine is a common sign of bladder cancer, even at early stage. The test cost of bloody urine is low and the test procedure is simple so that the bloody urine is tested initially. However, the bloody urine test has low specificity to bladder cancer and not effective since other conditions can also lead to blood in urine.
(2) Cytology test: Urine cytology test is a useful diagnostic tool in detection of cancer cells in urine. However, the method is not used widely due to low sensitivity. Moreover, the cytology test needs to be interpreted by a qualified pathologist and the detection cost is high.
(3) Cystoscopy: Cystoscopy has become a major diagnostic tool for bladder cancer. However, this procedure is invasive and the cost is quite high.

As to kidney cancer, it is hard to detect because there are few symptoms in the early stage of the disease. Approximately 17% patients were presented with metastatic disease at diagnosis of renal cell carcinoma from data in the SEER registry covering 2002 SEER registry covering 2002 through 2008. Thus periodic health examination should be the key to find kidney cancer at early stage. In clinic, few patients have symptoms of bloody urine, pain, abdominal masses, weight loss, anemia, fever, etc. But most of symptoms are shown at late stage. The most common tool for diagnosis of kidney cancer is sonography and in combination with other medical imaging examinations such as intravenous injection of contrast media, urography, computed tomography, or magnetic resonance imaging (MRI), etc.

The inventor of the present invention uses proteomic techniques to compare changes in urine protein profile caused by urinary tract cancers and find out TACSTD2 (Tumor-associated calcium signal transducer 2) protein whose concentration changes have significant meanings. Thus TACSTD2 protein can be used as target molecules for detection of bladder cancer and kidney cancer. Urine is collected as samples for tests. The sample collection is non-invasive and the test procedure is easy with lower risk.

The correlation between the urinary TACSTD2 protein of the present invention and bladder cancer/or kidney cancer have not been mentioned in article or patent available now. Although patients with certain cancers such as ovarian cancer have higher expression level of TACSTD2. TACSTD2 expressed by the lesion of ovarian cancer may also be released into urine. TACSTD2 used in the screening of urinary tract cancers still has high reliability. According to the patient's medical history and other symptoms together with the medical tests for detection of urinary tract cancers clinically such as urine cytology, cystoscopy, intravenous urography, X-ray, ultrasonic examination, computed tomography, or MRI. Thus the accuracy of the method of the present invention that predicts the presence and progression of urinary tract cancers by urine protein TACSTD2 can be increased dramatically.

### SUMMARY OF THE INVENTION

It is a primary object of the present invention to provide a urinary biomarker for urinary tract cancers and applications of the same that provide a non-invasive way to assess the likelihood of a person having urinary tract cancer, the degree of tumor invasion into surrounding tissues, or the grade/malignancy of cancer cells so as to monitor disease progression, to find out optimal treatment against the disease, and to improve treatment results.

It is another object of the present invention to provide a urinary biomarker for urinary tract cancers and applications of the same that can be used together with detection methods available now including urine occult blood test, detection of other biomarker molecules, cytological test or medical imaging examinations (cystoscopy), etc. for assessment of the risk of urinary tract cancers, the degree of tumor invasion or the grade/malignancy of tumor cells.

In order to achieve the above objects, a urinary biomarker for urinary tract cancers according to the present invention is used. The biomarker including TACSTD2 protein is present in urine samples of the subjects. The higher amount of the biomarker in the urine represents that the subject has a higher risk to have urinary tract cancers, the worse the tumor progression, or the tumor cells have higher grade. The biomarker can be applied to screen urinary tract cancers or diagnose urinary tract cancers at early stage. The accuracy of the tumor assessment and diagnosis is further improved with reference to medical history, symptoms, and other examination methods available now.

A method for assessment of urinary tract cancers of the present invention includes following steps. First provide a urine sample of a subject. Then detect expression level of TACSTD2 protein in the urine sample of the subject quantitatively. TACSTD2 protein is the urinary biomarker. Next compare the expression level of TACSTD2 protein of the subject with the expression level of TACSTD2 protein of the control. Finally, assess the risk of the patient having urinary tract cancers, the degree of tumor invasion, or the malignancy/grade of cancer cells according to the comparison result. Urine samples of health people without bladder cancer and kidney cancer or urine samples of the subjects collected before are used in the control group. When the expression level of the subject is higher than that of the control and the difference therebetween is larger, it is predicted that the higher the risk of patients having urinary tract cancers, the worse the tumor invasion (extension to surrounding tissues) or the higher grade/malignancy of tumor cells.

A test kit for detecting urinary tract cancers of the present invention is further provided. The test kit includes at least one test reagent developed based on the method for assessment of urinary tract cancers mentioned above.

Among the urinary tract cancers mentioned above, the assessment of bladder cancer or kidney cancer has better effect.

The purpose, technique, features and functions of the present invention are described in details by following embodiments and figures.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a flow chart showing steps of an embodiment of a method for assessing urinary tract cancers according to the present invention;

FIG. 2 is a graph showing test results of protein TACSTD2 in urine samples of 48 patients/subjects by LC-MRM/MS method of an embodiment according to the present invention;

FIG. 3 is a ROC curve for TACSTD2 protein in original urine samples of 277 patients according to the present invention;

FIG. 4A shows quantitative detection results of TACSTD2 protein in urine samples detected by using ELISA test according to the present invention;

FIG. 4B is a ROC curve obtained by comparing TACSTD2 protein in urine samples of a hernia patient group with that of a group of patients with bladder cancer according to the present invention;

FIG. 4C is a ROC curve obtained by comparing TACSTD2 protein in urine sample of a group of patients with LgEs bladder cancer with that of a group of patients with hernia according to the present invention;

FIG. 5A shows TACSTD2 protein detection results of urine samples of hernia patients, in urine of patients with bladder cancer, and in cell lysate of a bladder carcinoma cell line of an embodiment according to the present invention;

FIG. 5B and FIG. 5C show TACSTD2 protein detection results of urine samples of 10 hernia patients, 5 patients with LgEs bladder cancer, 5 patients with HgEs bladder cancer, and 6 patients with HgAs bladder cancer of an embodiment according to the present invention;

FIG. 5D shows quantitative results of TACSTD2 protein in urine samples of 10 hernia patients, 5 patients with LgEs bladder cancer, 5 patients with HgEs bladder cancer, and 6 patients with HgAs bladder cancer detected by Western blot analysis of an embodiment according to the present invention.

### DETAILED DESCRIPTION OF THE PREFERRED EMBODIMENT

The present invention uses proteomic techniques to find out TACSTD2 protein used as a urinary biomarker for assessment of urinary tract cancers so as to assist diagnosis of urinary tract cancer, assess disease progression and evaluate malignancy of cancer cells.

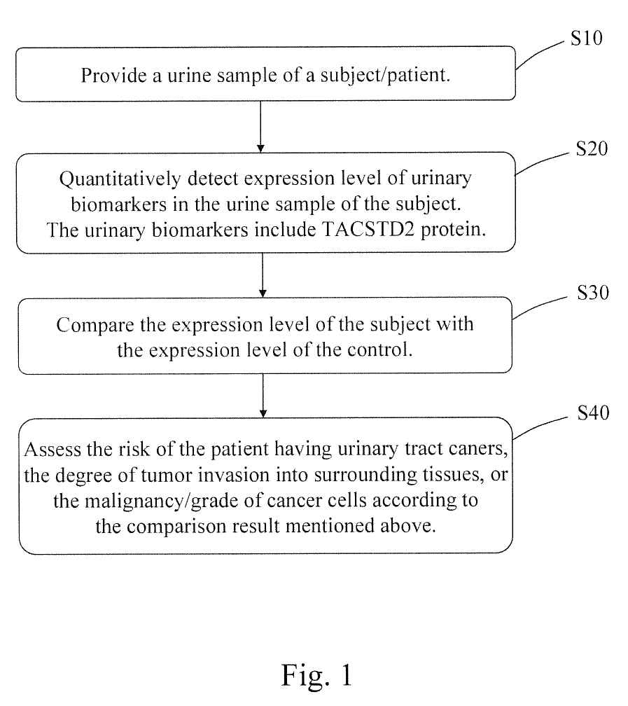

Refer to FIG. 1, a method for assessment of urinary tract cancers according to the present invention includes following steps:

Refer to the step S10, provide a urine sample of a subject/patient.

Then run the step S20, quantitatively detect expression level of urinary biomarkers in the urine sample of the subject. The urinary biomarkers include TACSTD2 protein (Tumor-associated calcium signal transducer 2, also called TACD2 or Trop-2). The TACSTD2 protein includes amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7 or their combinations.

As shown in the step S30, compare the expression level of the subject with the expression level of the control. The expression level of the control is obtained by quantitative detection of TACSTD2 in urine samples of a control group. Urine samples of health people without urinary tract cancers or urine samples of the subjects collected before are used in the control group.

Lastly, as shown in the step S40, assess the risk of the patient having urinary tract cancers, the degree of tumor invasion into surrounding tissues, or the malignancy/grade of cancer cells according to the comparison result mentioned above. When the expression level of the subject is higher than the expression level of the control and the difference therebetween is larger, it is assessed that the higher risk the subject has urinary tract cancers, the worse the disease gets or the more malignant the tumor is/the higher grade the tumor has. When the expression level of the control is set as 2.43 ng/mL and the expression level of the subject is lower than this value, the result shows that the subject is a non-cancer patient. When the expression level of the subject is higher than this value, this represents that the subject is a patient with at least low grade and early stage (LgEs) urinary tract cancer. Moreover, when the expression level of the control is set as 2.47 ng/mL and the expression level of the subject is lower than this value, it is predicted that the subject is a non-cancer patient. When the expression level of the subject is higher than this value, it is predicted that the subject is a patient with urinary tract cancer. According to the above two values of the expression level of the control, the subject is classified as a non-cancer patient when the expression level of the subject is lower than 2.43 ng/mL. When the expression level of the subject is ranging from 2.43 ng/mL and 2.47 ng/mL, the subject is assessed as a patient with LgEs urinary tract cancer. When the expression level of the subject is higher than 2.47 ng/mL, the subject is categorized as the patient with urinary tract cancer.

In the present invention, various quantitative techniques including Western blot analysis, enzyme-linked immunosorbent assay (ELISA), mass spectrometry (MS), etc. has been used to detect the amount of TACSTD2 protein in the urine sample of respective subject and also used to verify feasibility of the biomarker in screening, early detection, disease progression monitoring, and assessment of malignancy of urinary tract cancers. Moreover, the biomarker of the present invention can be used together with other tests for diagnosis of urinary tract cancers available now to improve the accuracy of the assessment. In practice, other techniques including the antibody detection, chemiluminescence detection, fluorescence detection, or chromatography can also be used in detection.

The purposes, functions and principles of the present invention are described in details in the following embodiment.

Procedure 1: collect urine.

Get urine samples from a control group of non-cancer patients and patients with bladder cancer by using protease inhibitor cocktail tablet and sodium azide (1 mM).

Procedure 2: concentrate the urine samples by ultracentrifugation.

Purify protein particles in urine by using ultracentrifugation. In brief, 12.5 ml urine sample is melted at 4° C. and the sample is centrifuged at 17,000×g for 30 minutes (4° C.) for removing large cells and debris. After centrifugation, the supernatant is centrifuged again at 100,000×g for 70 mins at 4° C. in a Beckman L8-80M ultracentrifuge so as to precipitate vesicles corresponding to the particles. The precipitate obtained is put in a centrifuge tube, washed by 5 ml phosphate buffered saline (PBS) for eliminating polluted protein and centrifuged at 100,000×g for 70 min at 4° C. in a Hitachi CS150 GXL micro ultracentrifuge. Then remove the supernatant and particles are suspended in 50 μl PBS. After vacuum drying, add 5 μL lysis buffer (10 mM Tris-HCl, 1 mM EDTA, 1 mM EGTA, 50 mM NaCl, 50 mM sodium fluoride, 20 mM sodium pyrophosphate, 1 mM Alloxan, and 1% Triton X-100) and 45 μL PBS into the tube. Then the tube is stored in ice for 15 min. After concentration, urine protein is detected by DC protein assay. Next the urine sample is stored at the temperature below 20° C. for following tests.

The step 10 of providing a urine sample is completed by the procedure 1 and procedure 2.

Procedure 3: Western Blot Analysis

Total urine proteins (100 μg) from individual samples were resolved on sodium dodecyl sulfate-polyacrylamide gel electrophoresis (SDS-PAGE) gels and transferred electrophoretically onto polyvinylidene fluoride (PVDF) membranes for biomarker verification. The membranes were blocked for 1 hour at room temperature with 5% nonfat dried milk in Tris-buffered saline with 0.1% Tween-20. Afterward, the membranes were probed using anti-TACSTD2 antibody at 1:500 overnight at 4° C. The membranes were probed with primary antibody followed by streptavidin-alkaline horseradish peroxidase-conjugated secondary antibody, and developed using enhanced chemiluminescence detection. The relative signal intensity of TACSTD2 protein detected in the blots was quantified using a computing densitometer.

Procedure 4: LC-MRM/MS analysis

A mass spectrometer (AB/MDS Sciex 5500 QTRAP) with a nanoelectrospray ionization source was used for all LC-MRM/MS analyses. All acquisition methods used the following parameters: ion spray voltage, 1900-2000 V; curtain gas setting, 20 psi (UHP nitrogen); interface heater temperature, 150° C.; and MS operating pressure, 3.5×10-5 Torn Q1 and Q3 were set to unit resolution (0.6-0.8 Da full width at half height). MRM acquisition methods were constructed using three MRM ion pairs per peptide with fragment-ion-specific tuned collision energy (CE) voltages and retention time constraints. A default collision cell exit potential of 35 V was used for all MRM ion pairs, and the scheduled MRM option was used for all data acquisition, with a target cycle time of 2 s and a 4-min MRM detection window. Transitions of 82 peptides (41 light peptides and 41 heavy peptides) corresponding to 29 target proteins were quantified in a LC-MRM/MS run.

Procedure 5: Sandwich ELISA

White 96 well polystyrene microtiter plates (Corning Corp., Corning, N.Y.) are coated with goat anti-TROP2 antibody (AF650, R&D, USA). By incubation at 4000 ng/mL in PBS (50 μL in each well) for 2 h. Theb the plates were blocked by the addition of 200 μL per well of bovine serum albumin (BSA) (Sigma) 1% in PBS overnight at 4° C. 50 μL urine protein from 81 hernia patients, 40 LgEs patients and 63 HgEs patients is diluted by the blocking buffer in a ratio of 1:2 and incubated for 1 h at room temperature. A recombinant TACSTD2 protein (650-T2, R&D, USA) is used as a standard. Subsequently, biotinylated anti-human TACSTD2 antibody (BAF650, R&D, USA) (1:50 dilution in PBS containing 1% BSA) is applied and incubated for an additional 1 h at room temperature. Then 50 μL streptavidin-alkaline phosphatase (RPN1234, Amersham bioscience, UK) (1:3000 dilution in PBS containing 1% BSA) is added and incubated for 40 min at room temperature. Next the substrate 4-methylumbelliferyl phosphate (Molecular Probes, Eugene, Oreg.) is diluted to 100 μM with an alkaline phosphatase buffer mixture (alkaline phosphatase buffer: PBS=1:2) and 100 μL is added to each well. The fluorescence is measured by a SpectraMax M5 microplate reader (Molecular Devices, Sunnyvale, Calif.) with excitation and emission wavelength set at 355 and 460 nm respectively.

The quantitatively detection of expression level of urinary biomarkers in the urine sample in the step S20 is completed by the procedure 3, procedure 4, or procedure 5.

Procedure 6: statistical analysis

The differences in urine protein concentration of different groups clinically are detected by LC-orbitrap-MS/MS or LC-MRM/MS techniques and then analyzed by Mann-Whitney Test, one of the most powerful nonparametric tests used for comparing different groups. Receiver Operator Characteristic (ROC) and Area-Under-the-Curve (AUC) are analyzed for a range of cut-off values so as to get an optimal cut-off value. The optimal cut-off is determined by Youden's index (J). The equation: J=1−(false positive rate+false negative rate)=1−[(1−sensitivity)+(1−specificity)]=sensitivity+specificity−1.

The expression level of the control in the step S30 can be calculated by the procedure 6 so as to determine cut-off values or reference values used in the step S40 for assessing whether the patient has urinary tract cancers, the degree of tumor invasion into surrounding tissues, and the malignancy of cancer cells.

LC-MRM/MS is used to verify presence of the urinary biomarker-TACSTD2 protein and the expression of TACSTD2 protein in tumor cells of urinary tract cancers is confirmed by immunohistochemistry (IHC).

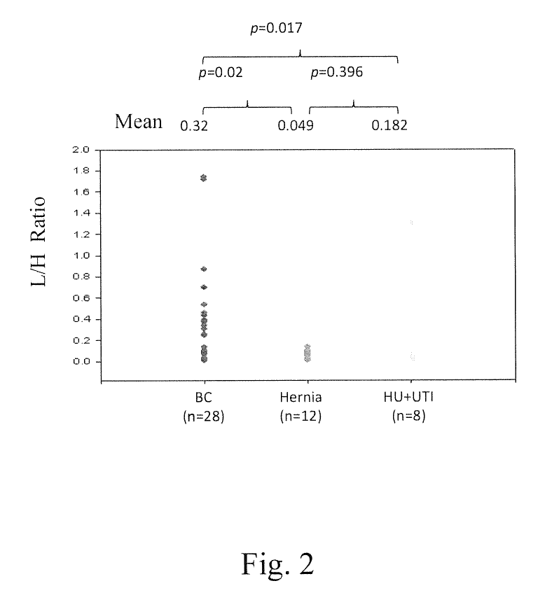

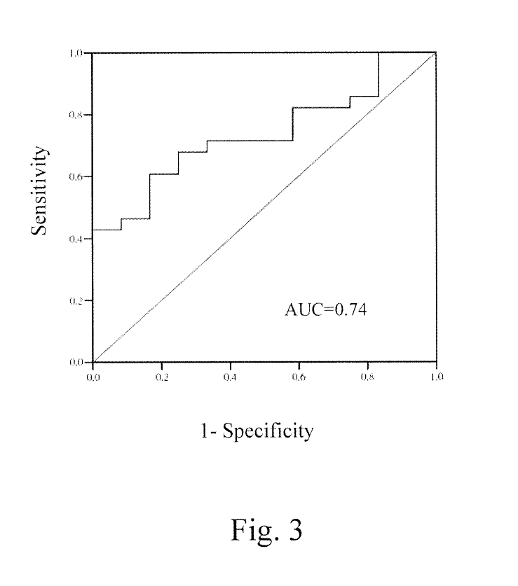

Refer to FIG. 2, the data of TACSTD2 protein of 48 samples detected by LC-MRM/MS is shown. The 48 samples are obtained from the test group of 28 patients with bladder cancer, the control group of 12 hernia patients and 8 urinary tract infection or hematuria (UTI/HU) patients (respectively represented by BC, Hernia, UTI+HU in figure). Each point in the figure shows average concentration and the p-value. The p-value on top (the first row) of the figure is obtained by comparing data of the BC group with that of the UTI/HU group. The p-value on the left side of the second row is calculated by comparing data of the BC group with that of the Hernia group while the p-value on the right side of the second row is obtained by comparing data of the Hernia group with that of the UTI/HU group. Compared with the data of the Hernia group and the UTI/HU group, the BC group, the expression level of the protein in urine samples of the patients with bladder cancer (BC) is high (p<0.05). Refer to FIG. 3, a ROC curve for the protein is revealed. The AUC value is 0.74. This means that the urinary biomarker of the present invention can be used to differentiate the group of the patients with bladder cancer (28) and the control group of the hernia patients (12).

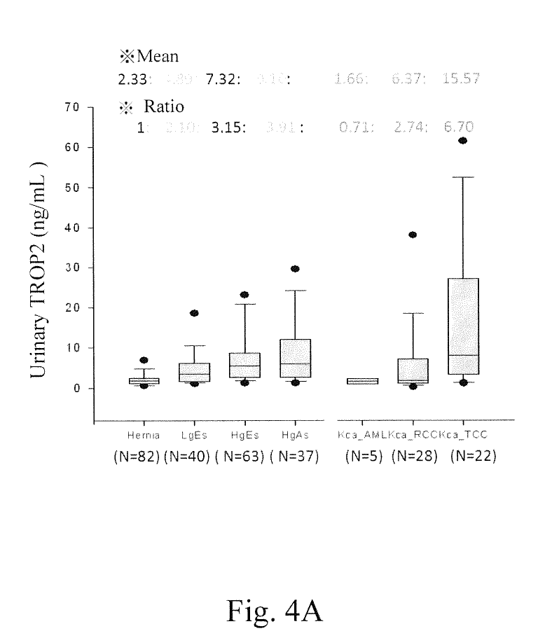

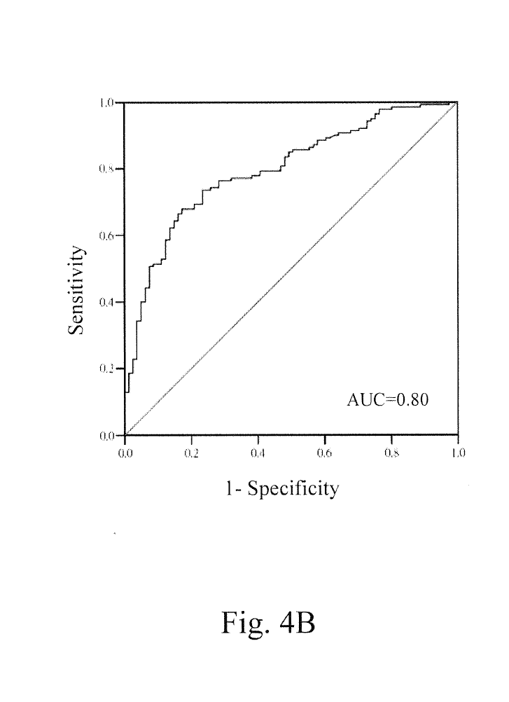

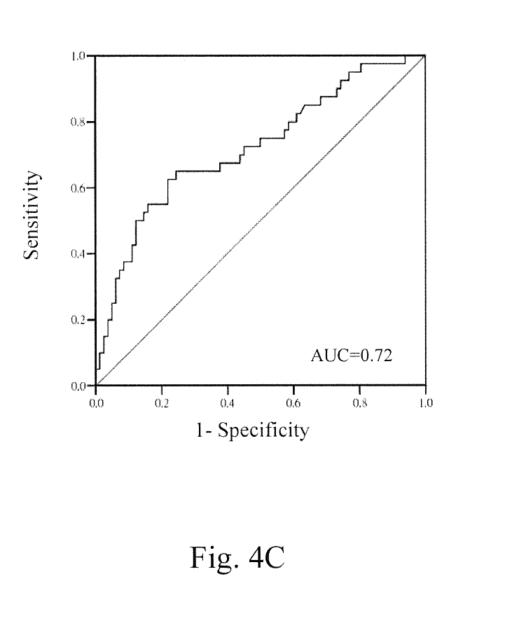

Refer to FIG. 4A, ELISA is used for quantitation of TACSTD2 protein in original urine samples of 277 samples. In the figure, Hernia, LgEs, HgEs, HgAs, Kca_AML, Kca_RCC and Kca_TCC respectively represent a group of patients with hernia, a group of patients with LgEs bladder cancer, a group of patients with HgEs bladder cancer, a group of patients with HgAs bladder cancer, a group of patients with renal angiomyolipoma (AML), a group of patients with renal cell carcinoma (RCC), and a group of patients with transitional cell carcinoma (TCC). The group of patients with renal angiomyolipoma (AML) is the control group of the patients with kidney cancer. The results show that the amount of TACSTD2 protein in urine samples of the group of patients with bladder cancer is about 2.1 to 3.9 times of that of the group of patients with hernia. Refer to FIG. 4B, a ROC curve obtained by comparing TACSTD2 protein in urine sample of the group of patients with hernia with that of the group of patients with bladder cancer is revealed. The AUC value is 0.80. This means the urine biomarker certainly can be used to differentiate the group of patient with hernia from the group of patients with bladder cancer. FIG. 4C shows a ROC curve obtained by comparing TACSTD2 protein in urine sample of the group of patients with LgEs bladder cancer with that of the group of patients with hernia is revealed. The AUC value is 0.72 and this means the urine biomarker actually can be used to differentiate the group of patient with LgEs bladder cancer from the group of patients with hernia. The TACSTD2 protein of the group of RCC patients is 4.9 times than that of the group of patients with AML and the TACSTD2 protein of the group of TCC patients is increased 10.4 times than that of the AML patients. The urinary biomarker certainly can be used to differentiate the AML patients from the patients with kidney cancer.

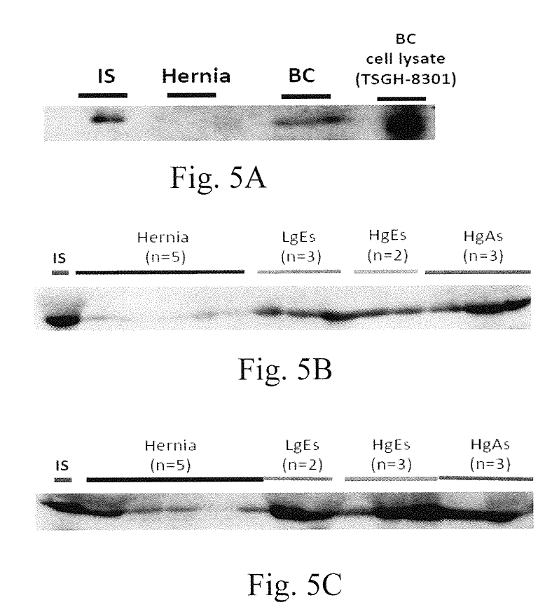

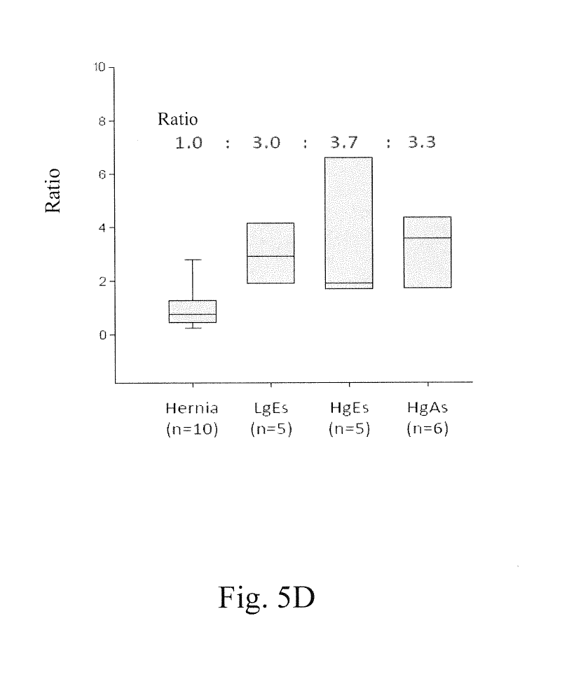

Refer to FIG. 5A, TACSTD2 protein detection results of urine samples of hernia patients and patients with bladder cancer, and in cell lysate of a bladder carcinoma cell line (TSGH 8301) are revealed. As to FIG. 5B and FIG. 5C, TACSTD2 protein detection results of urine samples of 10 hernia patients, 5 patients with LgEs bladder cancer, 5 patients with HgEs bladder cancer, and 6 patients with HgAs bladder cancer are disclosed. Refer to FIG. 5D, it shows quantitative results of TACSTD2 protein detected by Western blot analysis. In the above figures, hernia patients and patients with bladder cancer, and in cell lysate of a bladder carcinoma cell line (TSGH 8301) are respectively represented by Hernia, BC, and BC cell lysate (TSGH 8301) while LgEs, HgEs and HgAs respectively represent patients with LgEs bladder cancer, patients with HgEs bladder cancer, and patients with HgAs bladder cancer. As to “IS”, it is protein in urine made from HgAs bladder cancer cells used as internal standard for quantitative comparison.

ACSTD2 protein concentration in urine samples of bladder cancer patients is 2.1 to 3.9 times of that of the hernia patients. The average protein concentration of the hernia patients (control group) is 2.33 ng/mL while the average concentration of the patients with LgEs, HgEs and HgAs bladder cancer is 4.89 ng/mL, 7.32 ng/mL, and 9.10 ng/mL respectively. Thus it is learned that the concentration of ACSTD2 protein in urine samples of the patients is increasing along with tumor progression. Thus when the threshold value is set as 2.43 ng/mL, the subject whose protein concentration is over this value is considered as patient with at least LgEs urinary tract cancer. Otherwise, the subject is the patient without urinary tract cancer. The sensitivity and specificity of the performance of differentiation between the LgEs bladder cancer group and the control group are respectively 65.0% and 75.6% (p<0.001, AUC=0.72, n=121). When the threshold value is set as 2.47 ng/mL, the subject whose protein concentration is over this value is assessed as having urinary tract cancer otherwise is non-cancer. The sensitivity and specificity of the performance of differentiation between the LgEs bladder cancer group and the control group are respectively 73.6% and 76.5% while the positive predictive value 84.4% and the negative predictive value 62.6% (p<0.001, AUC=0.80, n=221). Moreover, there is a significant difference between TACSTD2 protein concentration in urine samples of low-grade patients and that of high-grade patients (p=0.014). Thus ACSTD2 protein can be used to differentiate the grade of the tumor.

The stage of bladder cancer means is a way of describing where the cancer is located and where it has invaded while the grade is used to describe how much the tumor cell looks like normal bladder tissue under a microscope including how well differentiated and growing speed. Low grade bladder cancer has cells that look like normal cells, well differentiated, growing slowly, and not likely to spread. High grade cancer cells look very abnormal, poorly differentiated, growing quickly and more likely to spread. At early stage, grade is one thing that the doctor takes into account when deciding the treatment way. If the tumor is high grade, further treatment is required to prevent the cancer recurrence. Early bladder cancer is also called non-muscle invasive bladder cancer or superficial bladder cancer. The cancer cells are only in the lining, the inner most layer of the bladder. The tumor is removed clearly by cystoscope or surgery. When tumor have invaded connective tissue of the muscle layer, it's advanced-stage bladder cancer or invasive bladder cancer. The active treatment includes removing a part of or the whole bladder together with postoperative radiotherapy or total removal of the bladder, radical cystectomy, with urinary diversion.

In the above step, the patients are categorized into the non-cancer group and the three bladder cancer groups. The bladder cancer groups include a group of low grade with early stage (LgEs), a group of high grade with early stage (HgEs) and a group of high grade with advanced stage (HgAs). According to the above results, it shows that the concentration of TACSTD2 protein in the urine samples of the high-grade patients is obviously increased (p<0.05, the difference between the two groups is statistically significant) after comparing the concentration of TACSTD2 protein in the urine samples of non-bladder-cancer patients with that of the bladder cancer patients, low grade patients with high grade patients, and early stage patients with advanced stage patients. The patients with kidney cancer also have similar results. According to the measured results of another embodiment of the present invention, the amount of TACSTD2 protein in the urine samples of kidney cancer patients is 3.8 to 9.4 times of that of the patients with benign renal diseases. Thus TACSTD2 protein certainly can be used as a non-invasive urine biomarker for assessment of urinary tract cancers. Moreover, this urinary biomarker has high specificity and high sensitivity so that the likelihood of a person having urinary tract cancers can be assessed effectively and the screening efficiency of urinary tract cancers is improved. Thus urinary tract cancers can be diagnosed and treated at early stage. The biomarker can also be used to assess the grade of the tumor and monitor disease progression. Therefore optimal treatment against the disease is used and treatment results are improved.

For applications in the future, the urinary biomarker of the present invention is used together with the methods available now such as urine occult blood test, detection of other biomarker molecules (NMP22), cystoscopy, various medical imaging examinations and cytological tests so as to confirm the type of cancer the patient has, check the degree of tumor invasion and the malignancy of cancer cells.

Moreover, a test reagent can be developed based on the above method for assessment of urinary tract cancers and a test kit including the test reagent is further provided for early diagnosis and effective assessment of urinary tract cancers.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A urinary biomarker for urinary tract cancers comprising a TACSTD2 protein; wherein the urinary biomarker in a urine sample of a subject is used for assessment of risk of the subject having at least one urinary tract cancer, progression of the urinary tract cancer, or malignancy of the urinary tract cancer.

2. The urinary biomarker as claimed in claim 1, wherein the TACSTD2 protein includes amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7 or their combinations.

3. The urinary biomarker as claimed in claim 1, wherein the urinary tract cancer is bladder cancer or kidney cancer.

4. A method for assessment of urinary tract cancers comprising the steps of:
providing a urine sample of a subject;
quantifying a subject expression level of a urinary biomarker in the urine sample of the subject and the urinary biomarker including a TACSTD2 protein;
comparing the subject expression level with a control expression level; and
assessing risk of the subject having at least one urinary tract cancer, progression of the urinary tract cancer, or malignancy of the urinary tract cancer according to the subject expression level and the control expression level.

5. The method as claimed in claim 4, wherein TACSTD2 protein includes amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7 or their combinations.

6. The method as claimed in claim 4, wherein the urinary tract cancer is bladder cancer or kidney cancer.

7. The method as claimed in claim 4, wherein the control expression level is obtained by quantifying the urinary biomarker in a urine sample of a control without the urinary tract cancer.

8. The method as claimed in claim 4, wherein the control expression level is obtained by quantifying the urinary biomarker in a previous urine sample of the subject.

9. The method as claimed in claim 4, wherein, the higher the risk of the subject having the urinary tract cancer, the worse the urinary tract cancer progression, or higher malignancy the urinary tract cancer when the subject expression level is higher than the control expression level and the difference therebetween is larger.

10. The method as claimed in claim 4, wherein the control expression level is 2.43 ng/mL; the subject is assessed as without the urinary tract cancer when the subject expression level is lower than the control expression level otherwise the subject is assessed having at least the urinary tract cancer with low grade and early stage (LgEs).

11. The method as claimed in claim 4, wherein the subject is assessed as without the urinary tract cancer when the control expression level is 2.47 ng/mL and the subject expression level is lower than the control expression level otherwise the subject is assessed as having the urinary tract cancer.

12. The method as claimed in claim 4, wherein the subject expression level and the control expression level are detected by Western blot analysis, mass spectrometry, antibody detection, chemiluminescence detection, fluorescence detection, enzyme-linked immunosorbent assay (ELISA) or chromatography.

13. The method as claimed in claim 4, wherein the method is used together with urine occult blood test, biomarker detection, medical imaging examinations including cystoscopy or cytological tests for assessing likelihood of the subject having the urinary tract cancer, progression of the urinary tract cancer, or malignancy of the urinary tract cancer.

14. The method as claimed in claim 4, wherein a test kit that assesses urinary tract cancers by urinary biomarkers includes at least one test reagent for detection of the urinary biomarker for the urinary tract cancer is developed based on the method for assessment of urinary tract cancers.
